(19) United States
(12) Patent Application Publication
(10) Pub. No.: US 2009/0227892 A1
(43) Pub. Date: Sep. 10, 2009

(54) Catheter Device for Percutaneous Procedures

(76) Inventors: Gabriele Krombach, Aachen (DE); Arno Bucker, Duren (DE); Joachim G. Pfeffer, Aachen (DE); Michael Friebe, Recklinghausen (DE)

Correspondence Address: CANTOR COLBURN, LLP, 20 Church Street, 22nd Floor, Hartford, CT, 06103, US

(21) Appl. No.: 12/300,418

(22) PCT Filed: May 11, 2006
(86) PCT No.: PCT/EP06/04429

§ 371 Date: Nov. 11, 2008

(51) Int. Cl. A61M 25/00; A61B 10/06; A61B 18/18

(52) U.S. Cl. 600/564; 604/117; 606/41

(57) ABSTRACT

The invention relates to a catheter device (1) for percutaneous interventions, in particular for injections, biopsies or the like, with an outer catheter (2) and with a tool for the intervention, in particular an injection needle (3), biopsy forceps, electrodes or the like, at the proximal end (4) of the catheter device (1). To be able to perform a percutaneous intervention free of injury, the tool is received in the outer catheter (2) in the delivery state, and the tip of the tool is recessed in the outer catheter (2) in the delivery state or aligned with the proximal end of the outer catheter (2), and the tool can be moved relative to the outer catheter (2) in such a way that at least the tip of the tool protrudes past the proximal end (4) of the outer catheter (2) in the state of engagement.

The invention relates to a catheter device for percutaneous procedures, especially for injections, biopsies, or the like.

Progress in molecular biology and genetic engineering in recent years has led to the development of various therapeutic substances directed toward angioneogenesis in an ischemically damaged myocardium or settlement of omnipotent stem cells and differentiation into myocytes in myocardial scars. Clinical studies in patients show that therapeutic tissue concentrations of such substances could only be achieved after direct injection into the tissue. An injection of such substances can only be easily conducted during open heart surgery. Such surgeries, however, are always associated with a not insignificant expense and corresponding risk. Percutaneous injection by means of a catheter device appears simpler in this context. However, a problem in this context is that an appropriate needle must be provided on the catheter during percutaneous injection and during introduction of the catheter into the body, for example, at the groin or bend of the arm, advancement up to the heart through the arteries must not lead to damage to the vessels. Another problem during percutaneous injection is that it can happen inadvertently that the heart tissue is also perforated during injection. Unintentional puncture of the heart tissue entails the danger of filling of the pericardium with blood and subsequent compression of the heart, with a possible fatal outcome.

Similar problems occur, for example, in percutaneous tissue sampling, in which biopsy forceps are introduced to the sampling site by means of a catheter.

The object of the present invention is to provide a catheter device for percutaneous procedures, in which the above-mentioned drawbacks are avoided.

To solve the above-mentioned task, a catheter device for percutaneous procedures, especially for injections, biopsies, or the like, is proposed that has an outer catheter and a tool for the procedure. The tool, for example, can be an injection needle, biopsy forceps, or electrodes. The aforementioned list, however, is not exhaustive, so other tools can also be included. In the case of the catheter device according to the invention, the tool is provided at the proximal end of the catheter device. It is now essential that the tool—in a feed state—be accommodated in the outer catheter. The tool tip of the tool is then fully within the outer catheter, i.e., the tool tip is embedded into it, i.e., retracted with respect to the outer catheter or aligned flush with the proximal end of the outer catheter, and in no case protruding. In this context, the insertion state describes the state in which the outer catheter, together with the tool, is introduced into the body and advanced to the procedure site. When the procedure site is reached, the catheter device is in the procedure state. It is now important in regard to the invention that the tool for the procedure can be moved with respect to the outer catheter extensively enough that at least the tool tip protrudes beyond the proximal end of the outer catheter in the procedure state. The embodiment according to the invention, as a result, permits the tool to be deployed from a retracted state for the procedure at the procedure site to a predefined length and, as required, retracted into the outer catheter after the procedure, in order to avoid the danger of injury to the vessel during withdrawal of the catheter.

In a preferred embodiment of the present invention, an inner catheter situated within the outer catheter is provided for deployment of the tool from the outer catheter, which can be moved with respect to the outer catheter. The tool is then provided on the proximal end of the inner catheter, so that the tool can be deployed at the procedure site from the outer catheter. On the distal end of the catheter device, the inner catheter protrudes beyond the outer catheter, so that the inner catheter can be moved by corresponding means during the procedure.

When an inner catheter is used, it is expedient to fasten the tool to the inner catheter at its distal end. As an alternative, the tool, in different embodiments, can also be designed in one piece with the inner catheter.

In another embodiment, without an inner catheter, it is proposed that an advancement device be attached on the proximal end of the outer catheter for deployment of the tool, in which case the tool is fastened in turn to the advancement device. In a preferred variant of this embodiment, the advancement device has at least one spring. This can be a coil spring that is compressed in the feed state. In order to be able to deploy the tool from the retracted state, at least one trigger device to release the spring is provided in this embodiment. For practical purposes, this is a trigger wire that can be operated from the outside. A locking of the advancement device, both in the retracted state and in the deployed state, can also be provided in this embodiment, in order to avoid an unintentional deployment or retraction of the tool.

Precisely during percutaneous procedures on the heart, it can happen that during injections, the needle is pushed too deeply into the heart tissue or the heart tissue is even punctured. In order to eliminate this problem, a stop to limit the procedure depth of the tool during the procedure is provided on the tool and/or on the inner catheter. This stop ensures that a needle, for example, can penetrate into the corresponding tissue only over a precisely stipulated length.

A stop of the aforementioned type can be implemented in a variety of ways. In order to guarantee a procedure or puncture-depth limitation in every case, i.e., to rule out that the stop might also penetrate into the corresponding tissue, it is proposed in a preferred embodiment of the invention that the stop protrude beyond the outer catheter in the radial direction in the deployed procedure state, whereby the protrusion preferably amounts to a multiple of the outside diameter of the outer catheter. It can ultimately be ensured by means of such a large stop that neither the tool nor the outer or inner catheter can puncture the heart tissue.

In order to be able to bring the catheter device according to the invention to the procedure site even with such a large stop, the stop, during the feed state, has at least one elastic stop device located within the outer catheter. In the feed state, the stop device is then in a compressed state. During deployment of the tool, the stop device is then also deployed and opens outward in the radial direction. In the uncompressed state, the stop then preferably runs at roughly a right angle to the outer catheter.

A mesh or spiral, for example, can be provided as stop device. Several spring arms are particularly preferred.

Although it is possible, in principle, to design the stop or the single or multiple stop devices as separate components, it is of particular advantage to design the stop as one piece with the tool or the inner catheter. In this case, the inner catheter should then consist of a shape-memory material, especially Teflon. This also applies to the tool, which, however, can also consist of other materials with a shape-memory material [sic; should probably read “. . . materials with shape-memory properties”—translator's note].

An additional advance is if the tool consists of a diamagnetic or only slightly paramagnetic material. Teflon, Nitinol, or an appropriately solid polymer plastic is particularly suited for this context. Ultimately, MR suitability or compatibility of the catheter according to the invention is obtained in this case. Passive or active markers or corresponding coatings can be used to make the tool made from such an MRI-compatible material visible. It is also understood that the catheter device according to the invention is suitable not only for MRI, but also CT applications. Ultimately, the embodiment according to the invention easily permits the tool on the tip of the catheter device to be advanced from an insertion location, for example, in the groin, to the heart through arteries under MRI, CT or X-ray control and inserted there into the target region.

It was pointed out in the introduction that injuries can occur during advancement of the catheter, and also during the procedure in the target region if the catheter device is not handled appropriately. In order to be able to control the catheter device precisely, both during advancement through the vessels, and also during the procedure, a control device is provided to control the alignment of the proximal end of the outer catheter. By controlling the proximal end of the outer catheter, not only can feeding be improved, especially in branches of vessels, but the tool can also be positioned exactly at the prescribed site of the target region.

In a particularly simple embodiment of the invention, the control device has a thread or wire that can be operated from the outside, which is attached in the region of the proximal end of the outer catheter. By pulling on the thread or wire, the proximal end of the outer catheter can be controlled in its alignment, both during feeding through the vessel and at the target location. By pulling on the wire or thread, bending occurs close to the proximal end of the outer catheter. To achieve bending of the outer catheter in a simple manner, the wall of the outer catheter in the area of the proximal end is thinned or notched in at least one location.

In order to facilitate injection and prevent deflection and sliding of the catheter device during positioning on the tissue for the procedure, a branching connection for application of a vacuum is provided in the area of the distal end of the outer catheter. Firm suction and therefore fixation of the outer catheter on the tissue can then be produced by the vacuum.

Practical examples of the invention will be described below with reference to the drawings. In the drawings:

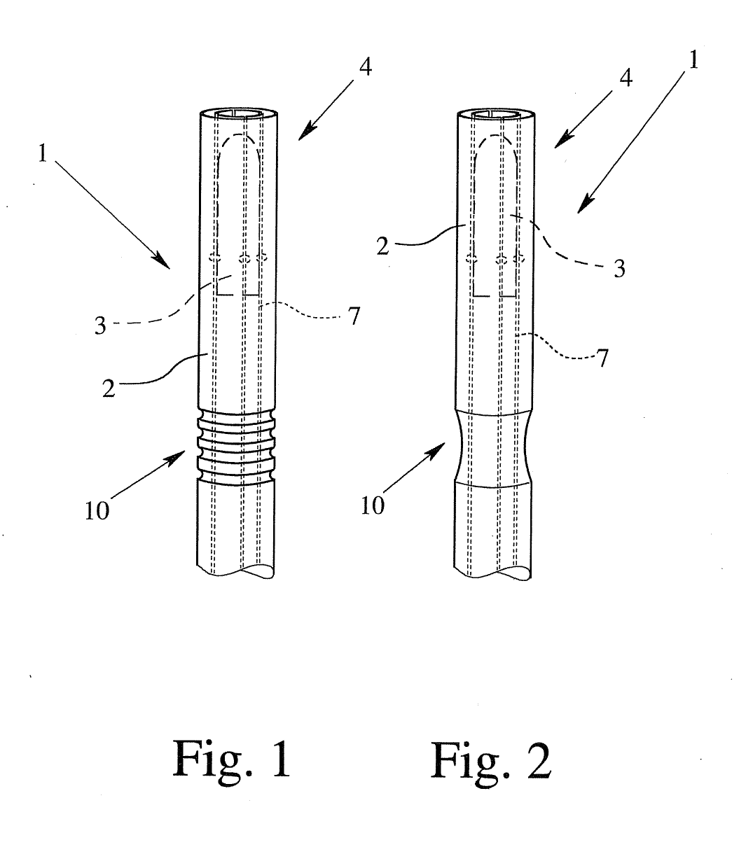

FIG. 1 shows a view of the proximal end of the catheter device according to the invention in the feed state,

FIG. 2 shows a view corresponding to FIG. 1 of another embodiment of the catheter device according to the invention,

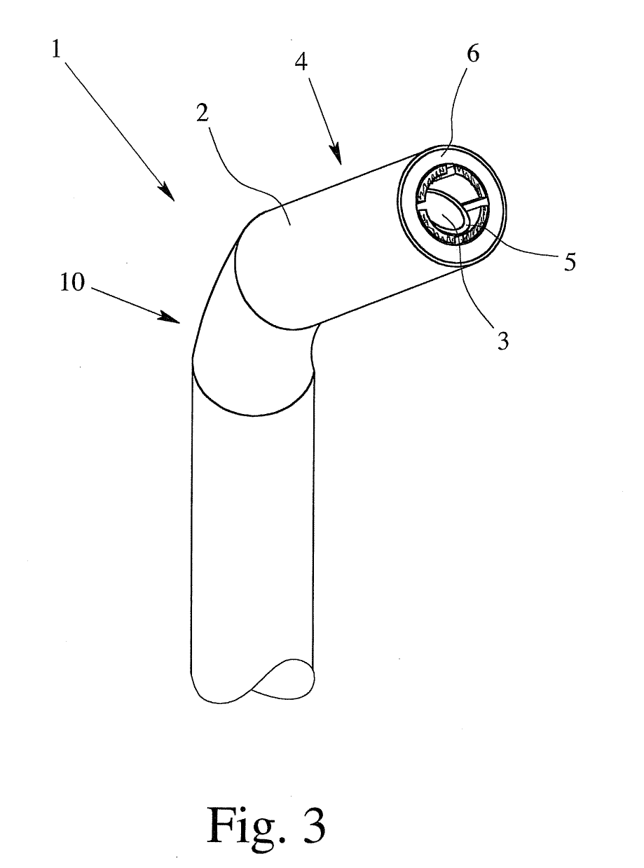

FIG. 3 shows a view of the catheter device of FIG. 2 with the bent proximal end in the feed state,

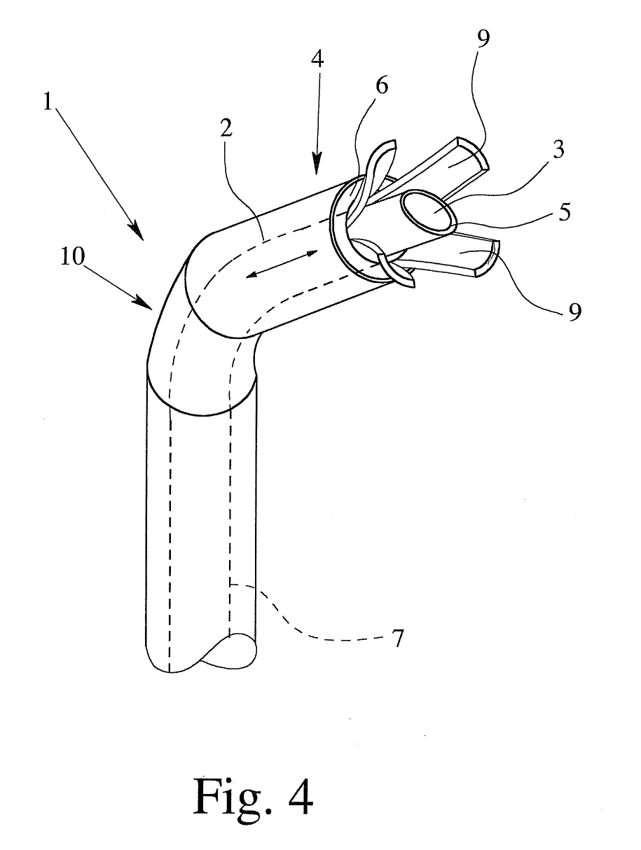

FIG. 4 shows a view corresponding to FIG. 3 of the catheter device from FIG. 3 with a partially deployed needle, and

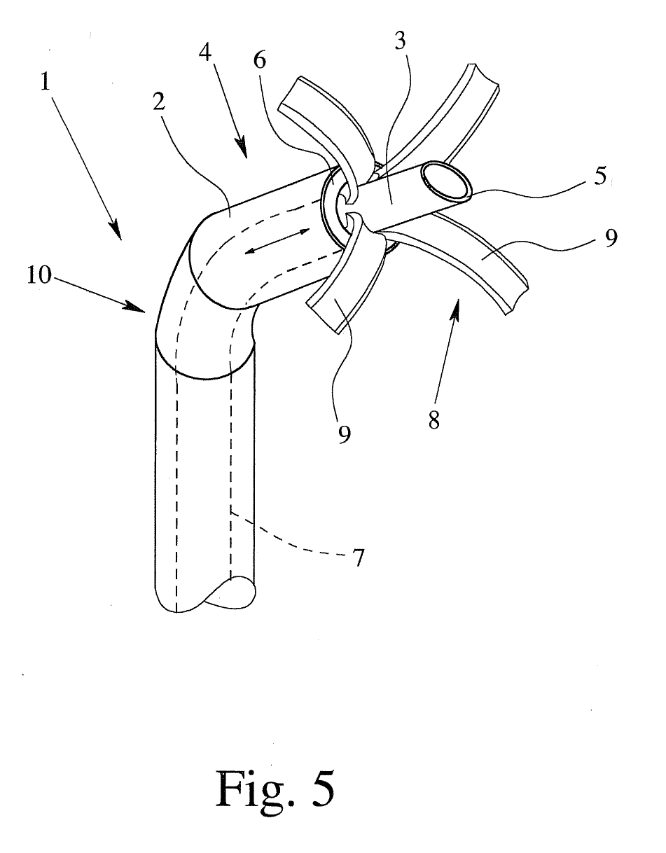

FIG. 5 shows a view corresponding to FIG. 4 of the catheter device with the deployed needle and the deployed stop in the injection state.

Two different embodiments of a catheter device 1 are shown in the drawings, which, however, differ only in the design of the bending site 10. Regardless of this difference, the catheter device 1 is designed for percutaneous or interventional procedures, such as injections. However, percutaneous procedures of a different type, for example, biopsies or the like are also possible. The catheter device 1 has an outer catheter 2 and a tool for the procedure. The tool in the present case is an injection needle 3, which is provided on the proximal end 4 of the catheter device 1. Depending on the type of procedure, it is understood that biopsy forceps, electrodes or the like, can also be provided, instead of an injection needle 3.

In the embodiments shown in FIGS. 1 to 3, the injection needle 3 is in a feed state in which the injection needle 3 is accommodated in the outer catheter 2 and the needle tip 5 is retracted in the outer catheter 2. In this case, the needle tip 5 does not protrude with its outer end beyond the front surface 6 of outer catheter 2. This can be seen especially from FIGS. 1 through 3. The injection needle 3 can be moved in the catheter device 1 with respect to the outer catheter 2, specifically, it can be deployed in such a way that at least the needle tip 5 in the procedure state, which is shown in FIG. 5, protrudes beyond the proximal end 4 of the outer catheter 2 and therefore the front surface 6. The mobility of the injection needle 3 with respect to the outer catheter 2 is then also such that not only is deployment possible, but the deployed injection needle 3 can also be retracted into the outer catheter 2 again, so that the state depicted in FIG. 3 is obtained.

For deployment of the injection needle 3 from the outer catheter 2, an inner catheter 7 is provided, which can be moved with respect to the outer catheter 2. The injection needle 3 is then attached on the proximal end of the inner catheter 7. In the present case, the injection needle 3 is inserted into the inner catheter with its rear distal end.

FIGS. 4 and 5, in particular, show that a stop 8 is provided on the inner catheter 7 for puncture-depth limitation of the injection needle 3 during injection. The stop 8, in the deployed procedure state as shown in FIG. 5, protrudes in the radial direction beyond the outer catheter 2. The radial extent of stop 8 is then several times larger than the outside diameter of the outer catheter 2. The stop 8 in the present case has a number of adjacent spring arms 9 as the stop device. The spring arms 9 are designed in one piece with the inner catheter 7. In order to produce the spring arms 9, the inner catheter is cut several times on its proximal end, so that the individual spring arms 9 are produced. In the present case, four spring arms 9 are provided, which are spaced about 90% [sic; should read as: 90°—translator's note] from each other in the uncompressed state. The uncompressed state of the spring arms is shown in FIG. 5. In the state depicted in FIGS. 1 to 3, the spring arms 9 are compressed and are situated within the outer catheter 2 in the feed state.

The inner catheter 7 in the present case, like the outer catheter 2, also consists of Teflon, which in this case involves a shape-memory material, which is important for the design of the spring arms 9 of the stop 8 with radial extent in the uncompressed state. The injection needle 5 in the present cases consists of Nitinol, i.e., a diamagnetic material, so the catheter device 1 is MRI-compatible. The injection needle 5, in principle, however, can also consist of another MRI-compatible material, such as non-ferromagnetic steel, another metal alloy, or a very strong plastic or carbon fiber.

It is not shown that a control device to control the alignment of the proximal end 4 of the outer catheter 2 is provided. The control device has a thread attached on the proximal end of the outer catheter 1, which is guided to the outside through the outer catheter 2. In this context, the thread runs parallel to the inner catheter 7, but not through it. By pulling on the thread, bending of the proximal end 4 of outer catheter 2 is obtained from the state depicted in FIG. 2 to the state depicted in FIG. 3. Bending of the proximal end 4 is favored by the fact that, in the area of the proximal end 4, the wall thickness of the outer catheter 2 is thinned or notched. For this purpose, possible embodiments for producing a bending site 10 are proposed in FIGS. 1 and 2.

A percutaneous injection by means of the catheter device 1 now occurs in such a way that the catheter device 1 is advanced with its proximal end into the heart from an injection location, for example, in the groin or in the bend of the arm, through the arteries. This preferably occurs under MRI, CT, or X-ray control. While the catheter is moved within the vessel, both the injection needle 3 and the inner catheter 7 are situated within the outer catheter 2. The spring arms 9 of the stop 8 are compressed and enclose the needle 3. When the catheter device 1 has been advanced to the target location, the inner catheter 7 is pushed out from the outer catheter 2. The spring arms 9 of the stop 8 then uncoil outward. Deployment of the stop 8 therefore occurs, until the injection needle 3 is completely released, as shown in FIG. 5. The injection needle 3 can then be inserted into the tissue until further penetration is prevented by the spring arms 9 lying on the tissue. Injection can then occur. After injection, first the inner catheter 7 is pulled back into the outer catheter 2. The outer catheter 2 can then be withdrawn.

1-18. (canceled)

19. A catheter device for percutaneous procedures, with an outer catheter and a tool for the procedure, on the proximal end of the catheter device,
wherein the tool in the feed state is accommodated in the outer catheter and the tool tip of the tool in the feed state is retracted into the outer catheter or aligned flush with the proximal end of the outer catheter;
wherein the tool can be moved with respect to the outer catheter, so that at least the tool tip protrudes beyond the proximal end of the outer catheter in the procedure state;
wherein a stop is provided to limit the procedure depth of the tool during the procedure;
wherein the stop in the deployed procedure state protrudes in the radial direction beyond the outer catheter;
wherein the stop has at least one elastic stop device arranged within the outer catheter in the feed state, and the stop device deploys and opens outward during deployment of the tool; and
wherein the stop device is designed as spring arms, characterized in that the stop has an extent in the radial direction in the procedure state that is several times larger than the outside diameter of the outer catheter;
wherein the inner contour of the stop device corresponds to the outer contour of the tool in the area of the tool tip.

20. A catheter device according to claim 19, wherein the percutaneous procedures are selected from the group consisting of injections and biopsies; wherein the tool is selected from the group consisting of an injection needle, biopsy forceps, and electrode.

21. A catheter device according to claim 19, wherein for deployment of the tool from the outer catheter, an inner catheter that can be moved within the outer catheter with respect to the outer catheter is provided, and that the tool is provided on the proximal end of the inner catheter.

22. A catheter device according to claim 19, wherein the tool is attached with its distal end to the inner catheter or that the tool is designed in one piece with the inner catheter.

23. A catheter device according to claim 19, wherein for deployment of the tool, an advancement device is attached to the proximal end of the outer catheter, and that the tool is attached to the advancement device.

24. A catheter device according to claim 19, wherein the advancement device has at least one spring.

25. A catheter device according to claim 19, further comprising at least one triggering device to release the spring for deployment of the tool.

26. A catheter device according to claim 19, further comprising a trigger wire that can be operated from the outside, to release the spring.

27. A catheter device according to claim 19, wherein the stop is provided on the tool and/or the inner catheter.

28. A catheter device according to claim 19, wherein the stop is designed in one piece with the tool or the inner catheter.

29. A catheter device according to claim 19, wherein the inner catheter consists of a shape-memory material.

30. A catheter device according to claim 29, wherein the shape memory material comprises Teflon.

31. A catheter device according to claim 19, wherein the tool consists of a diamagnetic or only slightly paramagnetic material.

32. A catheter device according to claim 31, wherein the tool consists of Nitinol.

33. A catheter device according to claim 19, further comprising a control device to control alignment of the proximal end of the outer catheter.

34. A catheter device according to claim 33, wherein the control device has a thread or wire that can be operated from the outside, which is attached to the outer catheter in the area of the proximal end.

35. A catheter device according to claim 19, wherein the wall thickness of the outer catheter is thinned and/or notched to form a bending site in the area of the proximal end.

36. A catheter device according to claim 19, further comprising a branching connection to apply a vacuum in the area of the distal end of the outer catheter.
